(19) United States
(12) Patent Application Publication
(10) Pub. No.: US 2026/0158072 A1
(43) Pub. Date: Jun. 11, 2026

(54) COMPOSITION AND METHOD FOR PREVENTING OR TREATING OPTIC NEUROPATHY

(71) Applicant: ExoOne Bio. Co., Ltd., Taipei City (TW)

(72) Inventors: Tzou-Yien LIN, Taipei City (TW); Rong-Kung TSAI, Hualien City (TW); Yao-Tseng WEN, Hualien City (TW); Yi-Ju HSIEH, Taipei City (TW); Wei-Cheng TSAI, New Taipei City (TW); Zhi-Xiang LI, Taichung City (TW)

(21) Appl. No.: 19/410,840

(22) Filed: Dec. 5, 2025

Related U.S. Application Data
(60) Provisional application No. 63/728,427, filed on Dec. 5, 2024.

(51) Int. Cl. A61K 35/28; A61P 27/02

(52) U.S. Cl. CPC A61K 35/28; A61P 27/02

(57) ABSTRACT

Provided is a composition including an exosome and an excipient thereof and a method for preventing or treating optic neuropathy by administering the composition to a subject in need thereof.

### BACKGROUND

### Technical Field

The present disclosure relates to compositions comprising exosomes and their uses for preventing or treating optic neuropathy.

### Description of Related Art

Optic neuropathy results from damage to the optic nerve, which is a bundle of millions of fibers in the retina that sends visual signals to the brain. The optic nerve contains axons of nerve cells that emerge from the retina, leave the eye at the optic disc, and extend to the visual cortex where input from the eye is processed into vision. There are 1.2 million optic nerve fibers that derive from the retinal ganglion cells of the inner retina. Damage and death of these nerve cells, or neurons, lead to characteristic features of the optic neuropathy. The main symptom is loss of vision, with colors appearing subtly washed out in the affected eye. A pale disc is characteristic of long-standing optic neuropathy.

Many causes can lead to damage to optic nerve, including ischemic optic neuropathy, optic neuritis, traumatic optic neuropathy, and hereditary optic neuropathy. In ischemic optic neuropathies, there is insufficient blood flow (ischemia) to the optic nerve, which can be classified into anterior ischemic optic neuropathy (AION) and posterior ischemic optic neuropathy based on the location of the damage and the cause of reduced blood flow. On the other hand, optic neuritis is inflammation of the optic nerve, which is associated with swelling and destruction of the myelin sheath covering the optic nerve. Furthermore, optic nerves can be damaged when exposed to direct or indirect injury that leads to traumatic optic neuropathy. Direct optic nerve injuries are caused by trauma to the head or orbit that crosses normal tissue planes and disrupts the anatomy and function of the optic nerve, e.g., a bullet or forceps that physically injures the optic nerve. Indirect injuries, like blunt trauma to the forehead during a motor vehicle accident, transmit force to the optic nerve without transgressing tissue planes.

In addition, glaucoma also leads to damage of the optic nerve, where degeneration of axons of the retinal ganglion cells is a hallmark of glaucoma. Glaucoma causes vision loss if left untreated, and the loss of vision usually occurs slowly over a long period of time. Glaucoma is a leading cause of blindness in African Americans, Hispanic Americans, and Asians.

Current therapeutic strategies for optic neuropathy include systemic steroids, surgical decompression of the optic canal, combination therapy with steroids and surgery, and conservative observation. Steroids, often administered intravenously at high to mega doses (such as methylprednisolone), are a common treatment but come with a high risk of adverse effects, affecting up to 90% of users. These side effects range from infections and elevated blood sugar to severe complications like pancreatitis and adrenal insufficiency. Moreover, studies found no notable difference in visual outcomes among patients who underwent treatment with steroids, surgery, or conservative therapy. Both steroids and surgical decompression carry risks and have not shown clear benefits over spontaneous recovery.

Recently, the therapeutic use of mesenchymal stem cells (MSCs) in retinal diseases, optic nerve injury, and glaucoma has been extensively studied in various experimental models; however, safety concerns regarding MSC-based therapies remain controversial, as long-term follow-up studies have reported undesirable differentiation of transplanted MSCs.

The medical community, therefore, is still in search of methods and pharmaceutical compositions that prevent or treat optic neuropathy safely and effectively.

### SUMMARY

The present disclosure provides a method for preventing or treating optic neuropathy in a subject in need thereof, comprising administering to said subject a therapeutically effective amount of a composition comprising an exosome isolated from a mesenchymal stem cell and an excipient thereof, wherein the isolated exosome undergoes a refinement procedure.

In at least one embodiment, the refinement procedure comprises an ultrafiltration. In at least one embodiment, the ultrafiltration is carried out with a centrifugal filter device with a 100,000 molecular weight cutoff (MWCO) membrane. In at least one embodiment, the refinement procedure comprises at least one cycle of concentration and centrifugation. In at least one embodiment, the centrifugation is carried out at from 1,000×g to 20,000×g, e.g., about 1,000×g, about 2,000×g, about 3,000×g, about 4,000×g, about 5,000×g, about 8,000×g, about 10,000×g, about 12,000×g, about 15,000×g, about 18,000×g, or about 20,000. In at least one embodiment, the concentration is at least a 5-times concentration, e.g., at least a 5-times concentration, at least a 10-times concentration, at least a 15-times concentration, at least a 20-times concentration, at least a 25-times concentration, or at least a 30-times concentration. In at least one embodiment, the refinement procedure comprises reconstitution with Hank's balanced salt solution after concentration.

In at least one embodiment, the refined exosome reduces toxicity associated with administration of an exosome to an eye. In at least one embodiment, the administration is intravitreal injection.

In at least one embodiment, the refined exosome preserves retinal function in an eye, preserves visual function in an eye, provides neuroprotective effect to a retina in an eye, suppresses macrophage infiltration in an optic nerve in an eye, reduces inflammation in an optic nerve in an eye, and/or increases antioxidant activity in a retina in an eye.

In at least one embodiment, the optic neuropathy is ischemic optic neuropathy, inflammatory optic neuropathy, compressive optic neuropathy, infiltrative optic neuropathy, traumatic optic neuropathy, mitochondrial optic neuropathy, nutritional optic neuropathy, toxic optic neuropathy, hereditary optic neuropathy, radiation-induced optic neuropathy, optic nerve atrophy, glaucoma, or optic neuritis.

The present disclosure also provides a method for preparing the composition as described above for preventing or treating optic neuropathy, comprising obtaining an exosome isolated from a mesenchymal stem cell and refining the isolated exosome, wherein the refining comprises at least a step of ultrafiltration, reconstitution, centrifugation, and concentration. In another embodiment, the present disclosure also provides a composition comprising the exosomes prepared by the method described above and an excipient thereof.

### BRIEF DESCRIPTION OF THE DRAWINGS

By reading the following detailed descriptions of the embodiments and referring to the accompanying drawings, the present disclosure can be more fully understood.

FIG. 1 shows the experimental design for investigating neuroprotective effects of refined exosomes using an optic nerve crush (ONC) rat model.

FIGS. 2A to 2D show the comparison of original exosomes and refined UMSCs-Exosomes. FIG. 2A shows the experimental design; FIG. 2B shows the rat body weights at different time points; FIG. 2C shows the representative images of dark-adapted and light-adapted electroretinogram (ERG) waveforms at 3 cd·s/m²; FIG. 2D shows the quantitative analysis of dark-adapted a-wave and b-wave amplitudes and light-adapted a-wave and b-wave amplitudes. Data are represented as mean±SD in each group (* p<0.05, ** p<0.01, n=6). ns: not significant.

FIGS. 3A and 3B show the comparison of original exosomes and refined UMSCs-Exosomes. FIG. 3A shows representative images of retinal immunohistochemistry (IHC) staining, indicating Iba1-positive cells (upper panel, red), ED1-positive macrophages (middle panel, green), GFAP-positive astrocytes cells (lower panel, green), and DAPI-stained nuclei (blue). GCL: ganglion cell layer; IPL: inner plexiform layer; INL: inner nuclear layer; OPL: outer plexiform layer; ONL: outer nuclear layer. FIG. 3B shows the bar chart illustrating the number of Iba1+ cells, ED1+ cells, and percentage of GFAP immunoreactive area per high-power field, respectively. Data are presented as mean±SD in each group (ns: not significant; * p<0.05; ** p<0.01; *** p<0.001, n=6).

FIGS. 4A to 4D show the toxicity evaluation of refined UMSCs-exosomes after intravitreal injection in Wistar rats. FIG. 4A shows the experimental protocol; FIG. 4B shows the representative Fundus images and OCT images depicting ONH width and RNFL thickness at days 1, 2, 3, and 4 after intravitreal injection of refined UMSCs-exosomes. FIGS. 4C and 4D show the bar charts illustrating ONH width and average RFNL thickness, respectively. Data are presented as mean±SD in each group (ns: not significant, n=6).

FIGS. 5A to 5C show the toxicity evaluation of refined UMSCs-exosomes after intravitreal injection in Wistar rats. FIG. 5A shows the representative images of dark-adapted and light-adapted ERG waveforms at 3 cd·s/m². FIG. 5B shows the quantitative analysis of dark-adapted a-wave and b-wave amplitudes and light-adapted a-wave and b-wave amplitudes. FIG. 5C shows the representative images of retinal RIC staining showing ED1-positive macrophages (left panel, green), Iba1-positive cells (middle panel, red), CD8-positive cells (right panel, green), and DAPI-stained nuclei (blue). GCL: ganglion cell layer; IPL: inner plexiform layer; INL: inner nuclear layer; OPL: outer plexiform layer; ONL: outer nuclear layer. Data are presented as mean±SD in each group (ns: not significant, n=6).

FIGS. 6A and 6B show the effect of refined UMSCs-exosomes on flash visual-evoked potential (fVEP) recordings at 14 days after optic nerve crush. FIG. 6A shows the representative images of fVEP signals in each group; FIG. 6B shows the bar charts illustrating the P1-N2 amplitude of the different groups. Data are represented as mean±SD in each group (* p<0.05, n=6).

FIGS. 7A to 7F show the effect of refined UMSCs-exosomes on RGC density and apoptosis 14 days post-optic nerve crush. FIG. 7A shows the representative images depicting RGC density (white spots) in the central retina for each group. FIG. 7B shows the bar chart illustrating the number of RGC cells per mm² for each group. FIG. 7C shows the representative retinal images showing TUNEL-positive cells (green) contrasted with other cells (blue) in the retina. GCL: ganglion cell layer; IPL: inner plexiform layer; INL: inner nuclear layer. FIG. 7D shows the bar chart displaying the number of TUNEL-positive cells per field for each group. FIG. 7E shows the western blot analysis results of apoptosis-related markers Bcl-2, BAX, and cleaved caspase-3 (C. Caspase 3) protein expressions in rat retinas. FIG. 7F shows the bar chart of each group's relative protein expression of apoptosis-related markers Bcl-2, BAX, and cleaved caspase-3. Data are presented as mean±SD (* p<0.05; ** p<0.01, n=6).

FIGS. 8A to 8C show the retinal cross-sectional OCT image analysis. FIG. 8A shows the representative Fundus images, ONH width, and RFNL thickness profile of normal, PBS, and refined UMSCs-exosomes treatment groups with exosome at day-7 and day-14, respectively. FIGS. 8B and 8C show the bar charts illustrating the ONH width and average RFNL thickness, respectively. Data are represented as mean±SD in each group (* p<0.05; ** p<0.01, n=6).

FIGS. 9A to 9D show the effect of refined UMSCs-exosomes on macrophage infiltration in the optic nerve 14 days after optic nerve crush. FIG. 9A shows the representative images of optic nerve sections showing ED1-positive macrophages (green) and DAPI-stained nuclei (blue). FIG. 9B shows the quantification of ED1+ cells per high-power field in bar charts. FIG. 9C shows the western blot analysis results of CD206 and arginase expression in the rat optic nerve. FIG. 9D shows the relative protein expression levels of CD206 and arginase in bar charts. Data are presented as mean±SD (* p<0.05; ** p<0.01; *** p<0.001, n=6).

FIGS. 10A and 10B show the effect of refined UMSCs-exosomes on inflammation in the optic nerve 14 days post-optic nerve crush. FIG. 10A shows the western blot analysis results of iNOS, NF-kB, IL-1β, and Iba1 expressions in the rat optic nerve. FIG. 10B shows the relative protein expression levels of Iba1, IL-1β, NF-kB, and iNOS. Data are presented as mean±SD (* p<0.05; **p<0.01; *** p<0.001, n=6).

FIGS. 11A and 11B show the effect of refined UMSCs-exosomes on antioxidative stress proteins in retinal tissue 14 days post-optic nerve crush. FIG. 11A shows the western blot analysis of Nrf2, HO-1, and SOD2 expressions in the rat retina. FIG. 11B shows the relative protein expression levels of Nrf2, HO-1, and SOD2 in bar charts. Data are presented as mean±SD for each group (* p<0.05; ** p<0.01, n=6).

FIGS. 12A and 12B show that refined UMSCs-exosomes treatment activates the Nrf2 pathway through the involvement of both Akt and ERK. FIG. 12A shows the western blot analysis results of pAkt, Akt, pERK, and ERK expressions in the rat retina. FIG. 12B shows the relative protein expression levels of p-Akt/Akt, p-ERK/ERK in bar charts. Data are presented as mean±SD for each group (* p<0.05, n=6).

### DETAILED DESCRIPTION

The following examples are used for illustrating the present disclosure. A person skilled in the art can easily conceive the other effects of the present disclosure, based on the disclosure of the specification. It will be apparent that one or more embodiments may be practiced without specific details. The present disclosure can also be implemented or applied as described in different examples. It is possible to modify or alter the following examples for carrying out this disclosure without contravening its scope for different applications. Titles or subtitles may be used in this disclosure for the convenience of a reader, which shall have no influence on the scope of the present disclosure.

Extracellular vesicles are a broad term to describe all secreted membrane vesicles. As employed herein, the term includes exosomes, microvesicles (also referred to microparticles), ectosomes, matrix vesicles, calcifying vesicles, prostasomes, oncosomes, retrovirus-like particles, bacterial extracellular vesicles, intraluminal vesicles, and apoptotic bodies. Extracellular vesicles as employed herein include microvesicles and exosomes. Extracellular vesicles generally have a diameter in the range of from 10 nm to 5,000 nm. Microvesicles as employed herein refer to vesicles released after formation by budding from the cytomembrane and, for example, generally have a diameter in the range of from 100 nm to 1,000 nm. Exosomes are produced inside multivesicular bodies and are released after fusion of the multivesicular body with the cytomembrane. Generally, exosomes have a diameter in the range of from 30 nm to 200 nm.

Exosomes are a type of membrane-bound extracellular vesicles (EVs) produced in the endosomal compartment of most eukaryotic cells and some prokaryotic cells. These nano-sized vesicles generally have a diameter from 30 to 150 nm and are found to contain proteins, genetic materials, lipids, and metabolites. Depending on the cell types from which they are derived, exosomes can deliver diverse signals and involve in many physiological processes and functions such as development, proliferation, differentiation, immunomodulation, angiogenesis, and progression of different diseases. Therefore, exosomes hold promises as a tool to modulate physiological functions in an organism and thereby as a means to ameliorate symptoms and to prevent or treat diseases.

Exosomes have a natural negative charge, which can lead to non-specific interactions with retinal cells and trigger inflammatory responses, potentially causing toxicity when administered in an eye. Additionally, contamination from fetal bovine serum (FBS) can contribute to the toxic effects on the retina. During the refinement procedure of the present disclosure, Hank's balanced salt solution (HBSS) was used for multiple washes and resuspensions of exosomes, thereby reducing FBS and proteins. The presence of cations such as sodium, potassium, calcium, and magnesium in HBSS helps to neutralize exosomes' surface charge and exerts fewer toxic effects when administered in an eye.

In this disclosure, all terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the descriptions of the present disclosure. Thus, the terms used herein are defined based on the meaning of the terms together with the descriptions throughout the specification.

Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.” The term “about” means plus or minus 0.1% to 50%, 5% to 50%, 10% to 40%, 10% to 20%, or 10% to 15% of the number to which reference is being made.

As used in this disclosure, the singular forms “a,” “an,” and “the” include plural referents unless expressly and unequivocally limited to one referent. The term “or” is used interchangeably with the term “and/or” unless the context clearly indicates otherwise.

Also, when a part “includes” or “comprises” a component or a step, unless there is a particular description contrary thereto, the part can further include other components or other steps, not excluding the others.

As used herein, the phrase “at least one,” in reference to a list of one or more elements, should be understood to mean at least one element selected from any one or more of the elements in the list of elements, but not necessarily including at least one of each and every element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently “at least one of A and/or B”) can refer, in one embodiment, to at least one, optionally including more than one A, with no B present (and optionally including elements other than B); in another embodiment, to at least one, optionally including more than one B, with no A present (and optionally including elements other than A); in yet another embodiment, to at least one, optionally including more than one A, and at least one, optionally including more than one B (and optionally including other elements).

The phrase “an effective amount” refers to the amount of an active ingredient that is required to result in a reduction, inhibition, or prevention of a disorder or condition, or one or more symptoms of such condition or disorder in a subject. An effective amount will vary, as recognized by those skilled in the art, depending on routes of administration, excipient usage, and the possibility of co-usage with other therapeutic treatment.

As used herein, the term “composition” can be prepared according to any method known in the art for the manufacture of pharmaceuticals. Such composition or combination may contain sweetening agents, flavoring agents, coloring agents, and preserving agents. A formulation can be admixed with nontoxic and pharmaceutically acceptable excipients which are suitable for manufacture. Non-limiting formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, lozenges, packets, troches, elixirs, suspensions, solutions, syrups, soft and hard gelatin capsules, suppositories, sterilized injection fluid, packaged powder, on patches, in implants, etc.

As used herein, pharmaceutically acceptable carriers, including buffers, are well known in the art and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; physiological saline; sterilized water; isotonic agents; and/or non-ionic surfactants. See, e.g., Remington: The Science and Practice of Pharmacy 20^{th} Ed. (2000) Lippincott Williams and Wilkins, Ed. K. E. Hoover.

As used herein, the term “treat,” “treating,” or “treatment” refers to the application or administration of one or more active agents to a subject afflicted with a disorder, a symptom, or a condition of a disease, or a progression of the disease, with the purpose to cure, heal, relieve, alleviate, alter, remedy, ameliorate, improve, or affect the disorder, the symptom or the condition of the disease, the disabilities induced by the disease, or the progression of the disease.

As used herein, the term “preventing” or “prevention” refers to preventive or avoidance measures for a disease or symptoms or conditions of a disease, which include but are not limited to applying or administering one or more active agents to a subject who has not yet been diagnosed as a patient suffering from the disease or the symptoms or conditions of the disease but may be susceptible or prone to the disease. The purpose of the preventive measures is to avoid, prevent, or postpone the occurrence of the disease or the symptoms or conditions of the disease.

As used herein, the term “optic neuropathy” refers to a disease caused by damage to optic neuron tissues, such as optic nerve and retina, due to changes in intraocular pressure, lack of oxygen, oxidative stress, hyperglycemia, dry eye, infection, disturbances of metabolism, waste accumulation, and aging. For instance, optic neuropathy is intended to encompass all types of optic neuropathies known in the art. By way of example, the ocular disease may be optic nerve atrophy, glaucoma, open angle glaucoma, closed angle glaucoma, pigmentary glaucoma, optic neuritis, retinitis, keratitis, cataract, blepharitis, optic disc edema, optic neuromyelitis, or ischemic optic neuritis. As used herein, the term “optic neuropathy” encompasses all types of optic neuropathy, including ischemic optic neuropathy, inflammatory optic neuropathy, compressive optic neuropathy, infiltrative optic neuropathy, traumatic optic neuropathy, mitochondrial optic neuropathy, nutritional optic neuropathy, toxic optic neuropathy, hereditary optic neuropathy, radiation-induced optic neuropathy, glaucoma, or optic neuritis.

### EXAMPLES

Exemplary embodiments of the present disclosure are further described in the following examples, which should not be construed to limit the scope of the present disclosure. Materials and methods used in the procedures or experiments carried out in the following examples are described herein.

### Animals

Adult male Wistar rats, weighing 200 to 250 grams and aged 7 to 8 weeks, were obtained from BioLASCO Co., Taiwan. Male Royal College of Surgeons rats (RCS rats), weighing 70 to 80 grams and aged 3 to 4 weeks, were sourced from the Tzu Chi University animal center in Taiwan. Rats were kept in a controlled environment (23° C., 55% humidity, 12-hour light/dark cycle) in special cages with adequate food and water. An intramuscular injection of a combination of ketamine (100 mg/kg) and xylazine (10 mg/kg) was carried out for anesthesia. Additionally, 0.5% Alcaine (Alcon, USA) was applied for temporary ocular anesthesia. Animal care and experimental procedures were conducted following the guidelines of the Association for Research in Vision and Ophthalmology Statement for the Use of Animals in Ophthalmic and Vision Research, and the Institutional Animal Care and Use Committee (IACUC) at the Tzu Chi Medical Center approved all animal experiments.

### Exosome Preparation

Exosomes were prepared from the mesenchymal stem cells derived from human umbilical cord (umbilical cord mesenchymal stem cells, UCMSCs). Mesenchymal stem cells can be derived from human umbilical cord by any method known to a person skilled in the art, and the collected UCMSCs can be kept frozen until needed. To prepare exosomes from the frozen UCMSCs, the cells were thawed and cultured at 37° C. and 5% CO₂ in an incubator. After culturing for 96 hours, culture media were discarded, and the cells were washed. Fresh media were supplied to the UCMSCs and cultured for another 72 hours. Thereafter, culture media were discarded again, and serum-free culture media were supplied. The cells were cultured for another 48 hours, and the cell media were collected. These cell media collected were conditioned by the UCMSC culture and were referred to as conditioned medium (CM). The collected CM was first filtered by a 0.22 μm filter, and the exosomes in the CM were concentrated with a tangential flow filtration (TFF) cassette having a cutoff size of 30 to 300 kDa. The concentrated exosomes obtained were further proceeded with diafiltration by TFF with 10 times volume of saline. Finally, the obtained exosomes were sterilized by sterilizing filters.

The particle concentration and size distribution of the exosomes thus prepared were measured at 1.54×10¹⁰ particles/mL and 7.7×10⁹ particles/Eppendorf. Marker proteins including CD9, CD63, CD73, CD81, HSP70, TSG101, Calnexin, and Alix were identified through western blotting.

### Exosome Refinement

To further refine the exosome preparation, 1 mL of the original UCMSC-exosome solution was diluted with 9 mL of phosphate buffered saline (PBS). This mixture was ultrafiltered with Amicon Ultra-15 Centrifugal Filter Devices with a 100,000 Da molecular weight cutoff (MWCO) membrane (Merck Millipore, Billerica, MA, USA). The solution was concentrated to approximately 500 μL, following numerous cycles of centrifugation at 4000×g. The concentrate was then reconstituted with 10 mL of PBS and subjected to ultrafiltration again, concentrating it back to 500 μL. Finally, Hank's Balanced Salt Solution (HBSS, Thermo Fisher Scientific, USA) was added to the concentrate, and the solution was ultrafiltered with a 100,000 MWCO membrane, concentrating it to 500 μL. This final concentrate was preserved as the refined exosome preparation.

### Optic Nerve Crush Rat Model

To assess the neuroprotective effects of refined UMSCs-exosomes in an optic nerve crush (ONC) rat model, rats were divided into three groups: normal, PBS, and exosome, with each group consisting of six rats. Rats in the exosome group received an intravitreal injection of refined UMSCs-exosomes (5 μL) using a Hamilton syringe. Rats in the PBS group were injected with PBS, whereas rats in the normal group were not subjected to any intervention.

To carry out the optic nerve crush, a 60-gram microvascular clip (World Precision Instruments, FL, USA) was used. The ON buttons on both eyes were pressed for 30 seconds to crush the globe 0.5 mm posteriorly. In summary, rats were rendered unconscious with intramuscular injections of xylazine (10 mg/kg) and ketamine (100 mg/kg), following which the superior orbital border was incised. The optic nerve was exposed and subsequently crushed through self-closing jeweler's forceps for uniformity and reproducibility. Precise precautions were taken to prevent any harm to the ocular blood vessels. To make sure there was no blockage in the retinal circulation, an indirect ophthalmoscopy was conducted. The rats in the normal group were kept intact and did not receive optic nerve crush.

Subsequently, all the rats underwent optical coherence tomography (OCT) imaging on days 7 and 14 to assess the retinal structure. Retinal ganglion cell (RGC) density was measured using FluoroGold (Fluorochrome, LLC, Denver, CO, USA), and flash visual evoked potential (fVEP) measurements were employed to quantify retinal ganglion cell (RGC) density so as to evaluate overall visual function 14 days post-infarct. Terminal deoxynucleotidyl transferase dUTP nick end labeling (TUNEL) assay, immunohistochemistry (IHC), and immunoblot (western blot) analysis were also performed.

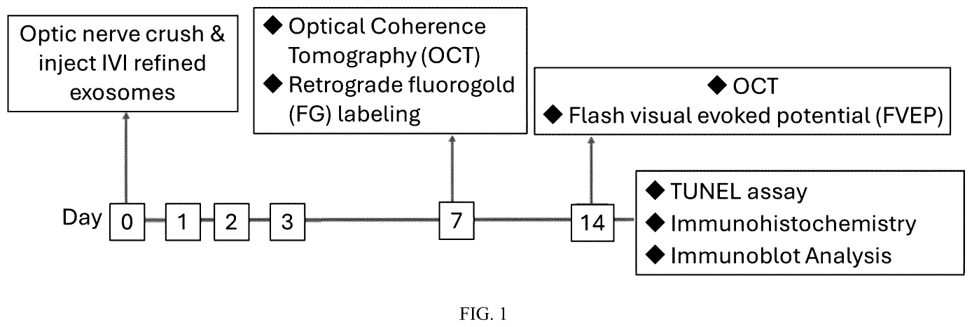

The experimental setup is visually represented in FIG. 1, and the distribution of rats among the groups is detailed in Table 1.

TABLE 1
Number of Wistar rats used in the ONC model

| Experiment | Normal | PBS | Exosome |
|---|---|---|---|
| Fluorogold | 3 | 3 | 3 |
| fVEP/OCT/IHC/TUNEL | 3 | 3 | 3 |
| Immunoblotting | 2 | 2 | 2 |

### Administration of Exosomes by Intravitreal Injection

Intravitreal injections were performed in the PBS and exosome groups after the crush. A 33-gauge needle (Hamilton 7747-01) was inserted under an operating microscope through the sclera at the ora serrata level using a Gaslight syringe (IA2-1701RN 10 μL SYR; Hamilton Co., Hamilton, KS, USA). The rats were injected with 5 μL of refined exosomes or phosphate-buffered saline (PBS) into their vitreous cavity. After injection, the site was pinched for 1 minute to prevent leakage of drugs.

Flash Visual Evoked Potentials (fVEP)

At 14 days following the exosome or PBS injection, flash visual evoked potentials (fVEP) measurements were conducted. To do this, after general anesthesia, the sagittal coordinates were exposed from the skull by opening about 1.5 to 2 cm with surgical scissors, and stereotaxic coordinates (AP=anterior-posterior; ML=medial-lateral; DV=dorsal-ventral) were marked on the skull to assist in the implantation of screws into the primary visual cortex region. The surface of the brain was perforated using a dental drill. Three electrodes were implanted in the primary visual cortex region of both hemispheres using stereotaxic coordinates (AP: −8 mm; ML: −3.0 mm). By using the Insight software (Diagnosys, MA, USA), the fVEPs were recorded. The electrode at the primary visual cortex acts as the active (positive) electrode, and the frontal cortex electrode is determined as the reference (negative) electrode. Further, the ground electrode was attached to the rat's tail. The fVEPs were measured without background illumination, utilizing a flash intensity of 30 cd s/m², and a single flash at a rate of 1.02 Hz. After averaging 64 sweeps, the results were recorded on a graph. The amplitude was derived from the fVEP assessments by measuring the difference between the highest point (P1 crest) and the lowest point (N2 trough). P1 crest is the highest point of the initial positive wavelet, and N2 trough is the lowest point of the subsequent negative wavelet.

### Optical Coherence Tomography (OCT)

Phoenix Micron IV microscope (Phoenix Research Labs, USA) with image-guided OCT was used to measure the retinal nerve fiber layer (RNFL) thickness and the optic nerve head (ONH) width (ONW). The OCT system featured a longitudinal resolution of 1.8 μm, a transverse resolution of 3 μm, a 3.2-mm field of view, and a 1.2-mm imaging depth within the retina. The anesthetic rat was administered with eye drops containing Alcaine, Mydrin-p, and methylcellulose. Next, the rat was positioned on the imaging frame at a suitable angle, so that the vertical light penetrates through the cornea properly. Circular scans around the optic disc were applied to visualize the RNFL. Simultaneously, a linear scan across the central portion of the optic disc was applied to visualize the width of Bruch's membrane opening ONW. This arrangement facilitated accurate measurements and imaging of the optic nerve area. At least three representative images were taken from each eye. ONH width and RNFL thickness were measured by “Insight” software, which creates layer segments and thickness profiles. GraphPad Prism was utilized to calculate the average RNFL thickness using the area under the curve method.

### Retinal Flat Mount Preparation and RGC Density Measurement

One week following the retrograde FluoroGold labeling, the rats were put to sleep, and the eyeballs were placed in 10% formalin for one hour. The retina was then meticulously removed from the eye and examined under a microscope. After meticulously scoping out the whole retina, four radial incisions were made to lay the retina flat on the microscopic slide with the vitreous side up.

For the analysis, a digital camera (Axiocam MRm), software (Axiovision 4.0), and a fluorescent microscope (Axioskop; Carl Zeiss Meditech Inc., Thornwood, NY, USA) with a 100× power and filters (excitation filter, 350 to 400 nm; barrier filter, 515 nm) were utilized. The RGC density of the central retina was determined by measuring the retinas at a distance of 1 mm from the optic nerve. RGC density was measured using Image Master 2D Platinum software.

### Sample Collection for Immunohistochemistry and TUNEL Assay

The optic nerve and eyeball were extracted from the entire eyes and placed in a solution of 4% paraformaldehyde, followed by storage at 4° C. After one day, needle syringes were used to puncture to extract the vitreous fluid. Subsequently, the cornea was incised to remove the eyes, including the optic nerve. The samples were transferred and immersed in a 30% sucrose-based solution and stored at 4° C. until they settled at the bottoms of the tubes. The optic nerves were cleaved from the eyes and immersed both the optic nerve and the eye in a mold containing an OCT compound. Special attention was given to position the samples to ensure precise tissue cross-section without any air bubbles. Subsequently, the molds were relocated to a liquid nitrogen container to solidify and then attentively placed in a −20° C. chamber for cutting. This gave thin slices (18 micrometers thick) showing the detailed part of the retina and optic nerve. On each slide, three different samples were placed for a thorough examination of the eye tissue. The slides were stored at −20° C.

### Immunohistochemistry (IHC)

The staining process started with the selection of ideal samples from the sectioned specimens, followed by a 10-minute heating at 37° C. Then, any remaining OCT gel was removed by using PBS wash. A hydrophobic barrier was created along the boundaries of the retina and the optic nerve cross-sections using an immerge marker. The tissues were then treated with a 2% bovine serum albumin (BSA) and 0.5% Triton in PBS at room temperature for 1 hour. Primary antibodies were prepared at a 1:100 dilution in 2% BSA and applied to the sample for overnight incubation at 4° C. Then, the tissue samples underwent three 5-minute washes with 1×PBS. Subsequently, secondary antibodies (1:200 dilution in PBST) were applied to the sample for 1 hour. After that, tissue samples were washed with PBS for 5 minutes. Mounting was carried out using Fluoroshield with 4′,6-diamidino-2-phenylindole (DAPI), with the addition of DAPI as a counterstain. Image capture was conducted using a fluorescence microscope with appropriate filters.

### TUNEL Assay

The TUNEL assay was used to detect apoptosis in retinal ganglion layer cells. Firstly, retinal cross-sections on microscope slides were washed three times with 1×PBS for 5 minutes each. Then, a liquid blocker was applied to mark the sample edges, ensuring the reagents remained contained during the procedure. Following this, the retinal cross-sections were exposed to 100 μL of Proteinase K solution (20 μg/mL) for 45 minutes, followed by a 10-minute incubation with 100 μL of Equilibrium Buffer at room temperature. Subsequently, a 50 μL terminal deoxynucleotidyl transferase (TdT) reaction mix, prepared according to Promega DeadEnd Fluorometric TUNEL System Kit instructions, was applied. The samples were then incubated with the TdT reaction mix for 1 hour at 37° C. in a humid chamber maintained with wet tissue paper. After incubation, the samples that underwent three 5-minute washes with PBS were counterstained with DAPI and mounted with Fluoroshield with DAPI. Finally, the mounted samples were stored at 4° C. until data collection, which was conducted using a confocal microscope equipped with appropriate filters. Positive signals were manually counted by the ImageJ software (National Institute of Health, USA). The TUNEL-positive cells in the ganglion cell layer of each section were counted in 10 high-power field (HPF, 400×), and an average of six sections per group was used for further statistical analysis.

### Western Blotting

Retina and optic nerve samples were homogenized using a radioimmunoprecipitation assay (RIPA) lysis buffer with protease and phosphatase inhibitors. Following centrifugation at 12,000×g for 20 minutes at 4° C., the supernatant was transferred to a new tube, and protein concentration was determined using a bicinchoninic acid (BCA) kit with BSA as the standard. For long-term storage, proteins were precipitated using trichloroacetic acid (TCA) acetone. In preparation for immunoblotting, precipitates were rehydrated, mixed with 4× loading dye containing a 10× reducing agent, denatured at 75° C. for 15 minutes, and cooled on ice for 5 minutes. Electrophoresis was performed by loading denatured protein samples onto a 4 to 12% Bis-Tris gel, running at 120 volts for 90 minutes. Triplicates of samples and 5 μL of BLUelf-prestained protein ladder (GeneTex, Irvine, CA, USA) were loaded. Proteins were transferred to a polyvinylidene difluoride (PVDF) membrane using Invitrogen's iBlot2 dry transfer device. Membranes were blocked in 1×TBST with 5% non-fat milk. Primary antibodies, prepared in 5% BSA in 1×TBST, were incubated overnight at 4° C. 3-actin served as an internal control. Membranes were washed in TBST and incubated with species-specific secondary antibodies conjugated with horseradish peroxidase (HRP) for 1 hour at room temperature. Using iBright FL1000 imaging equipment, the membrane was incubated in an electrochemiluminescence (ECL) complex (Immobilon Western Chemiluminescent HRP substrate) for 3 minutes. iBright Analysis software was used for result acquisition and quantification.

TABLE 2
List of antibodies used

| Antibody Name | Source | Cat. No. |
|---|---|---|
| Anti-Mannose Receptor (CD206) | Abcam, UK | ab64693 |
| Nf-κB p65 (D14E12) XP Rabbit mAb | Cell Signaling, USA | 8242s |
| Phospho-Akt (Ser473) (D9E) XP Rabbit mAb | Cell Signaling, USA | 4060 |
| Anti-iNOS, Rabbit polyclonal | Abcam, UK | ab3523 |
| β-Actin (8H10D10) Mouse mAb | Cell Signaling, USA | 3700 |
| BAX (D2E11) Rabbit mAb | Cell Signaling, USA | 5023 |
| BCL-2 Rabbit mAb | GeneTex, USA | GTX100064 |
| Cleaved Caspase-3 (Asp175) Rabbit mAb | Cell Signaling, USA | 9661 |
| Anti-IL-1β, Rabbit polyclonal | Abcam, UK | ab9722 |
| Anti-Nrf2, Rabbit polyclonal | Abcam, UK | ab137550 |
| Anti-Iba1 [EPR6136(2)], Rabbit monoclonal | Abcam, UK | ab178680 |
| Anti-HO-1 [EPR1390Y], Rabbit monoclonal | Abcam, UK | ab68477 |
| SOD2, Rabbit polyclonal | Invitrogen, USA | PA5-30604 |
| Phospho-p44/42 MAPK (Erk1/2) (Thr202/Tyr204) | Cell Signaling, USA | 9101 |
| Goat anti-rabbit HRP | Jackson ImmunoResearch, USA | 111-035-00 |

### Electroretinogram (ERG)

ERGs were performed to assess retinal functions. Firstly, animals were dark-adapted for at least 12 hours before the ERG recordings. The procedure involved placing a positive electrode gold wire loop on the cornea, a negative electrode beneath the animal's scalp, and a ground electrode into the tail (ColorDome Ganzfeld, Diagnosys LLC, Lowell, MA, USA), following the manufacturer's guidelines. A black cap was used to cover both the contralateral eye and the ERG equipment. Scotopic and photopic ERGs were recorded at 3 cd·s/m². Retinal photoreceptor and bipolar cell functions were evaluated by measuring the a- and b-waves, respectively.

### Statistical Analysis

All statistical analyses were performed by GraphPad Prism. Non-parametric Mann-Whitney and Kruskal-Wallis tests were employed for group comparisons. The results were reported as mean±standard deviation (SD). P-values less than 0.05 were considered statistically significant, with * p<0.05; ** p<0.01; *** p<0.001.

### Example 1: Refined UCMSCs-Exosomes Reduced Toxicity Associated with Original UCMSCs-Exosomes and Exhibited Superior Preservation of Retinal Function Compared to Original UCMSCs-Exosomes

In this experiment, Royal College of Surgeons (RCS) rats were categorized into the control group, the original exosome group, and the refined exosome group, with each group consisting of six rats. Each rat received a 3 μL intravitreal injection (IVI) as follows: the control group received PBS; the original exosome group received original UMSCs-exosomes without the refining process; and the refined UMSCs-exosomes group received refined UMSCs-exosomes. Body weight and physical appearance were monitored weekly for seven weeks. Electroretinography (ERG) was performed at the sixth week. At the end of the seven weeks, the rats were euthanized, and their eyes were collected for immunohistochemistry staining.

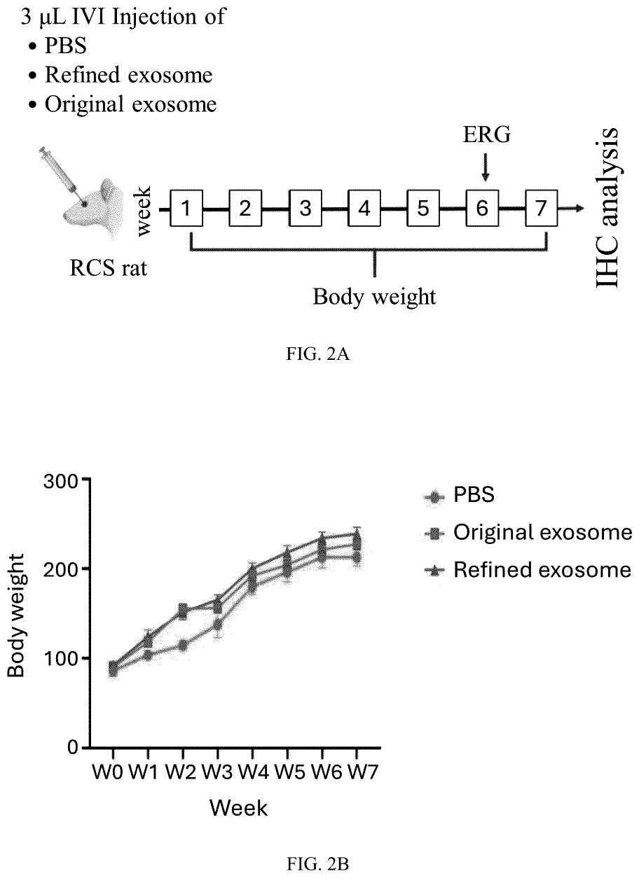

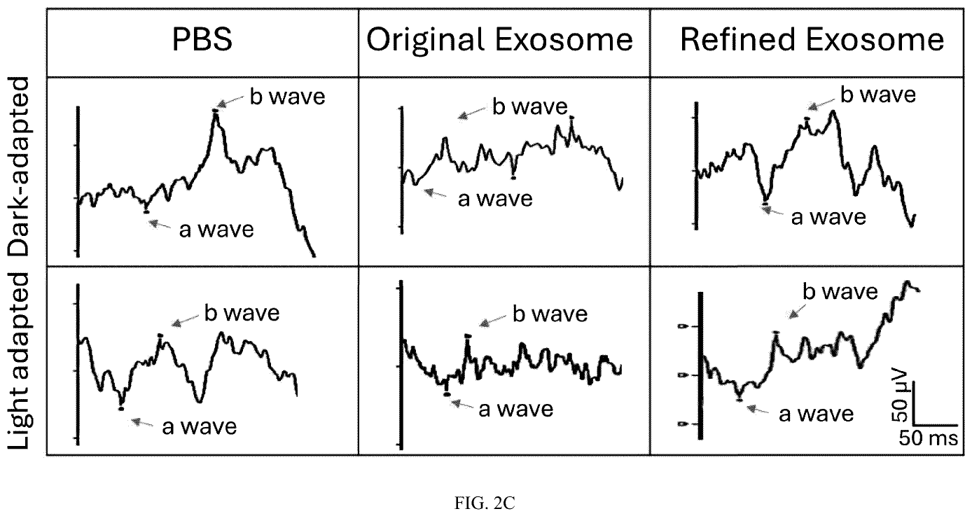

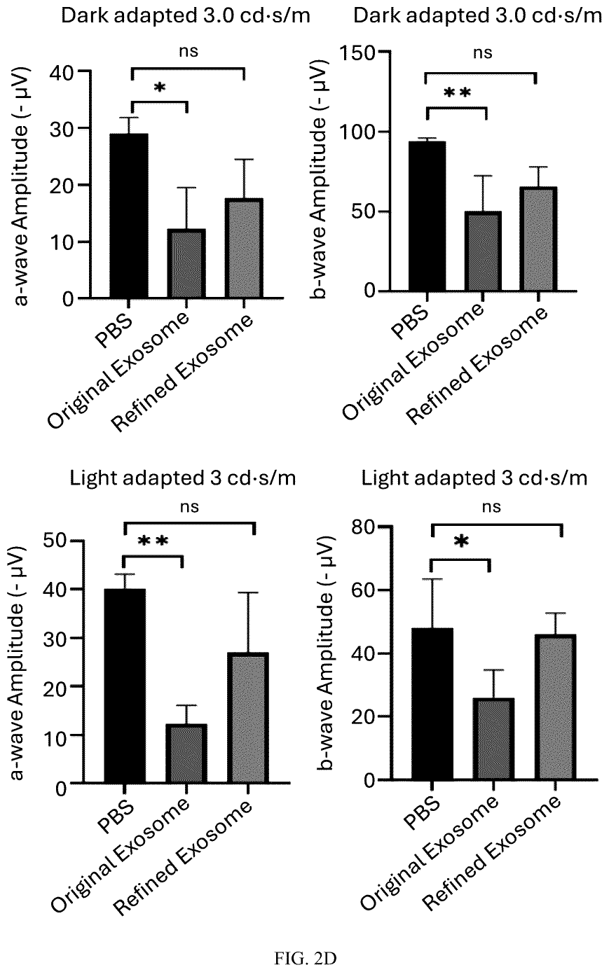

To compare the adverse effects of original UMSCs-exosomes and refined UMSCs-exosomes, 3 μL intravitreal injection (IVI) of each exosome type were administered into the RCS rats, with the control group receiving PBS. Rat body weight was monitored weekly for up to seven weeks, and electroretinography (ERG) evaluations were conducted at the sixth week (FIG. 2A). There were no obvious differences in the body weight of rats observed when compared to the PBS control group at various time points (FIG. 2B).

ERG analysis (FIGS. 2C and 2D) under dark- and light-adapted conditions (illuminance of 3 cd·s/m²) revealed that in dark-adapted conditions, the a-wave and b-wave amplitudes were reduced significantly by 1.65-fold (p=0.03) and 1.87-fold (p=0.006) in the original UMSCs-exosomes group compared to the PBS group. Under light-adapted conditions, the a-wave and b-wave amplitudes were reduced by 2.20-fold (p<0.01) and 1.77-fold (p=0.01) in the original UMSCs-exosomes group compared to the PBS group. These significant decreases in a-wave and b-wave amplitudes in the original UMSCs-exosomes group suggest that administration of original UMSCs-exosomes in the RCS rat eyes can have adverse effects on the retina.

Conversely, the refined UMSCs-exosomes group showed ERG responses similar to the PBS group, indicating that refining of exosomes as disclosed by the present disclosure reduces toxicity associated with UMSCs-exosomes administration.

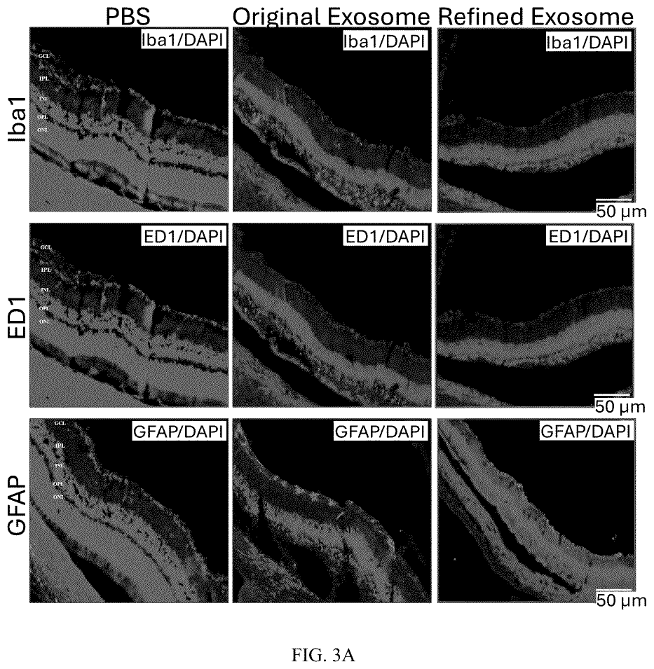

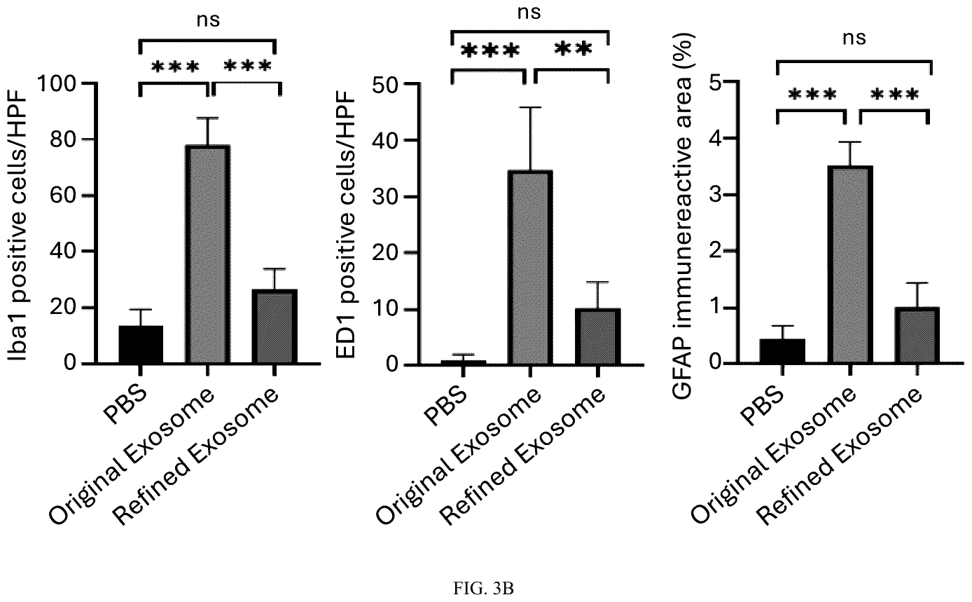

Furthermore, immunohistochemical (IHC) staining was performed to detect the presence of Iba1, ED1, and GFAP. RIC staining results (FIGS. 3A and B) showed significant increases in Iba1- and ED1-positive cells and glial fibrillary acidic protein (GFAP) immunoreactive areas in the original UMSCs-exosomes group compared to the PBS group. Specifically, Iba1-positive cells increased by 5.71-fold (p<0.001), ED1-positive cells increased by 11.55-fold (p<0.001), and GFAP immunoreactive areas increased by 8.07-fold (p<0.001). Conversely, the refined UMSCs-exosomes group exhibited significantly fewer Iba1-positive cells, ED1-positive cells, and GFAP immunoreactive areas compared to the original UMSCs-exosomes group. These results suggest that the refinement of UMSCs-exosomes may mitigate the potential toxicity associated with their administration, resulting in reduced neuroinflammation and better preservation of retinal function.

Overall, these findings suggest that refined UMSCs-exosomes offer superior preservation of retinal function and reduced adverse effects compared to original UMSCs-exosomes when intravitreally administered to RCS rats.

### Example 2: Safety Evaluation of Refined UMSCs-Exosomes for Intravitreal Injection in Wistar Rat Eyes

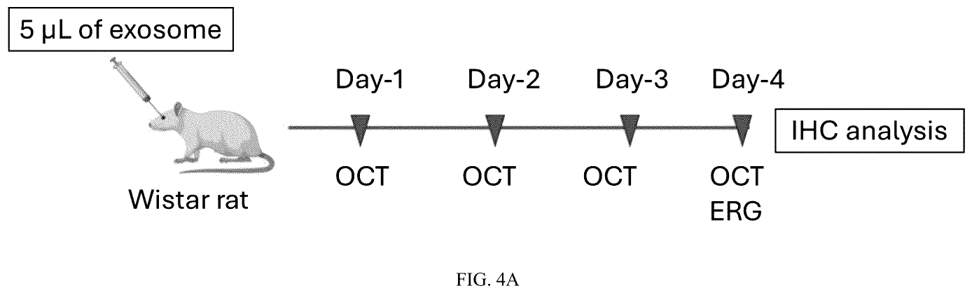

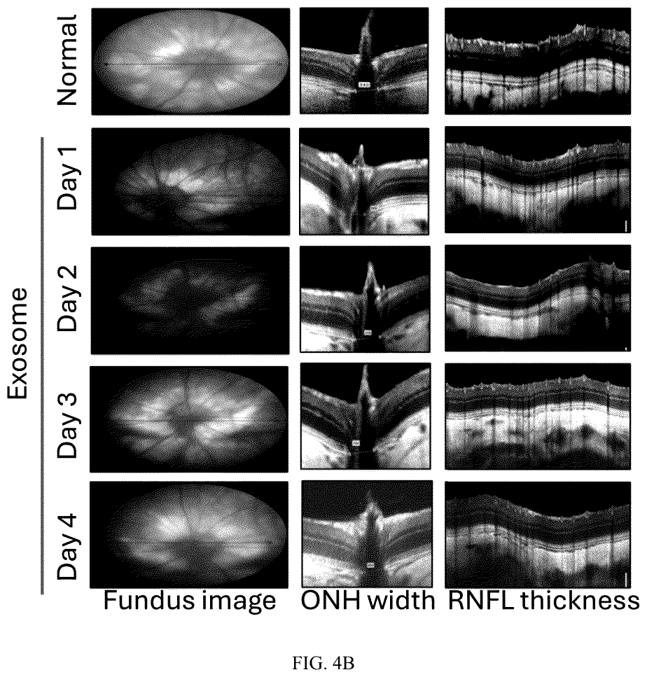

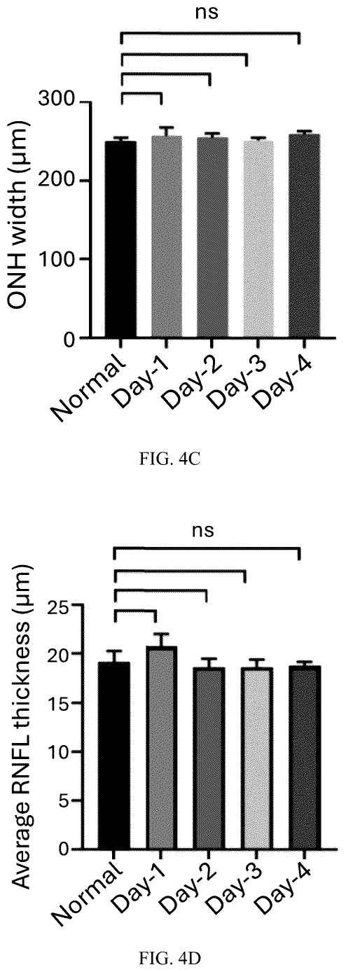

To assess the safety of refined UMSCs-exosomes, a 5 μL intravitreal injection was administered to Wistar rats. Optical coherence tomography (OCT) scans were utilized to monitor the optic nerve head (ONH) width and retinal nerve fiber layer (RNFL) thickness. On the fourth day post-injection, electroretinography (ERG) and immunohistochemical (IHC) analyses were performed. The results demonstrated that both ONH width and RNFL thickness remained stable throughout the observation period, indicating no structural impact from the treatment (FIGS. 4A to 4D).

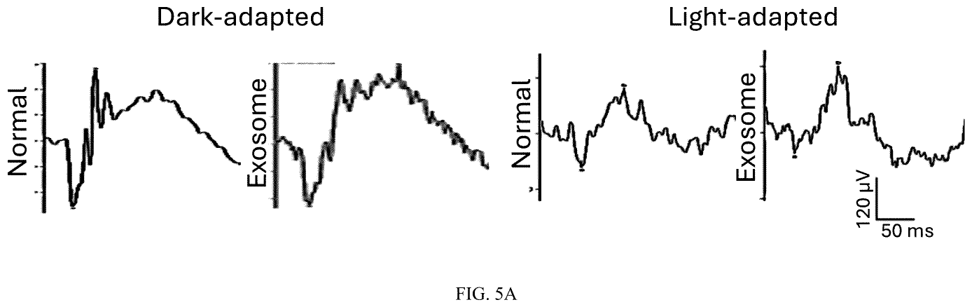

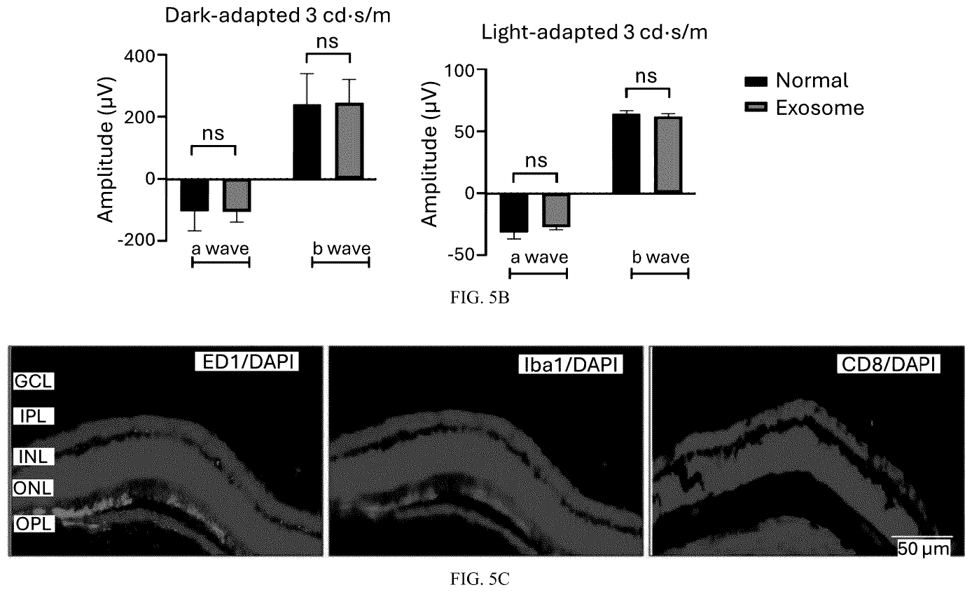

ERG analysis under both dark- and light-adapted conditions revealed no significant differences in a-wave and b-wave amplitudes between the refined UMSCs-exosomes group and the control group, suggesting no retinal toxicity (FIGS. 5A and 5B).

Furthermore, IHC staining showed no positive signals for Iba1, ED1, and GFAP, which are markers of neuroinflammation, indicating the absence of inflammatory responses (FIG. 5C).

Overall, these data suggest that intravitreal injection of refined UMSCs-exosomes does not cause toxicity in the Wistar rat retina, confirming their safety for intravitreal applications.

### Example 3: Refined UMSCs-Exosomes Preserve the Functional Visual Circuit

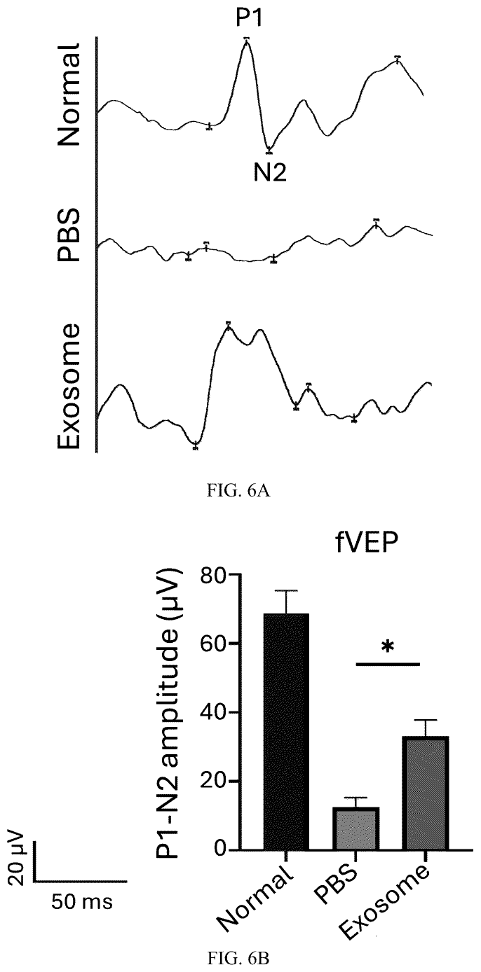

Flash visual-evoked potentials (fVEP) were performed to examine the visual function among the three groups of rats on day 14 in the ONC experiments (FIG. 6A). The P1-N2 amplitudes in normal, PBS-treated, refined UMSCs-exosomes-treated groups were 68.7±6.63 μV, 12.7±2.64 μV, and 33.2±4.49 μV, respectively (FIG. 6B). The refined UMSCs-exosomes-treated group exhibited a significant 2.6-fold increase in P1-N2 amplitude (p=0.028) compared to the PBS-treated group, indicating that refined UMSCs-exosomes treatment helps preserve the visual function.

### Example 4: Refined UMSCs-Exosomes Enhance RGC Survival Rate and Reduce RGC Apoptosis

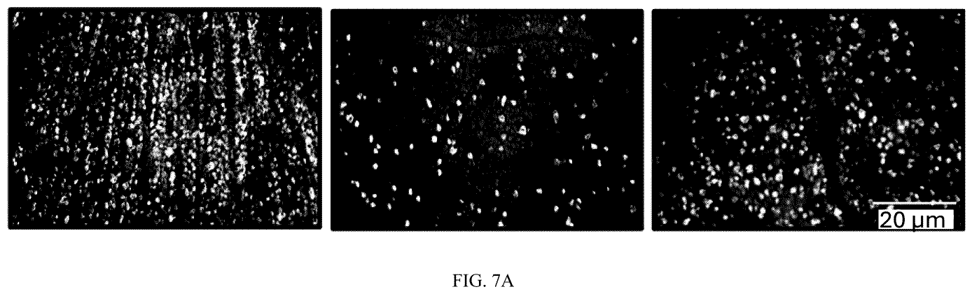

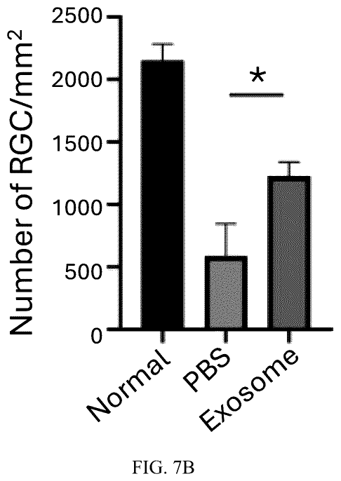

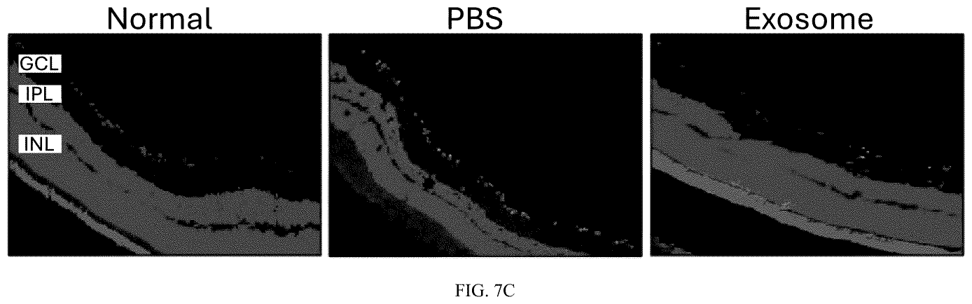

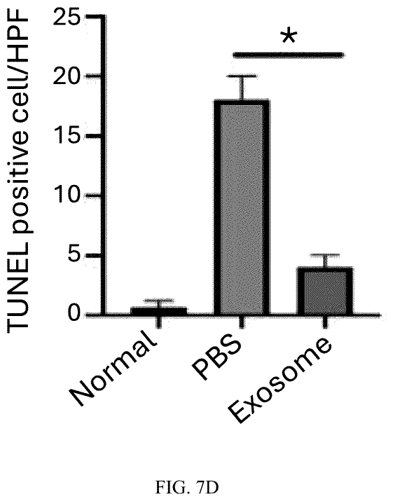

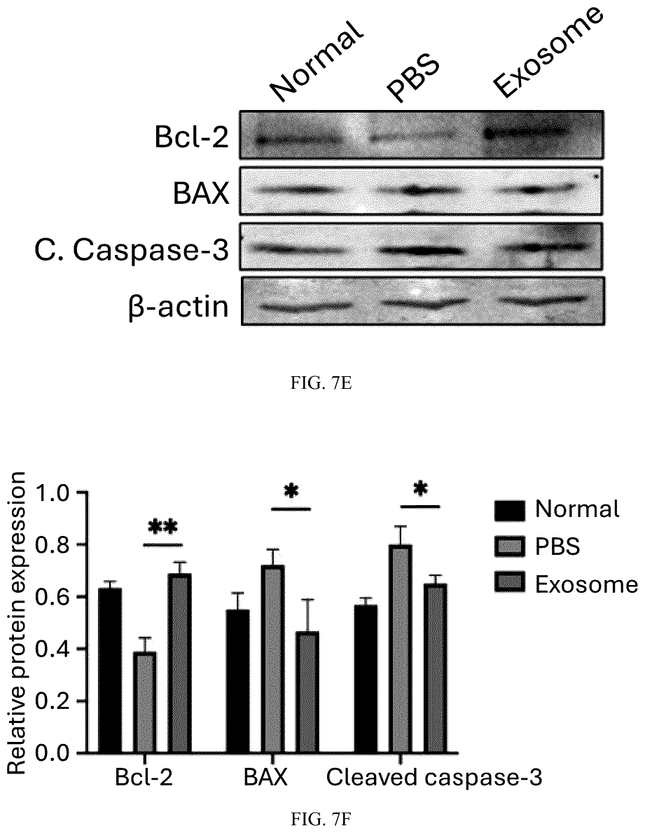

To assess the impact of refined UMSCs-exosomes on RGC survival, the RGC densities were counted in the central retinas of the rats in the three experimental groups (FIG. 7A). The RGC densities in normal, PBS-treated, and refined UMSCs-exosomes-treated groups were determined to be 2,153±128.9/mm², 585.7±261.5/mm², and 1,228±111.1/mm², respectively (FIG. 7B). The refined UMSCs-exosomes-treated group demonstrated a significant 2.1-fold increase in RGC density (p=0.011) compared to the PBS-treated group. These findings suggest that refined UMSCs-exosomes have the potential to protect RGCs from damage.

Additionally, a TUNEL assay was conducted to identify apoptotic RGCs across the RGC layer among the different experimental groups (FIG. 7C). The average numbers of TUNEL-positive cells per high-power field (HPF) in the RGC layer were 0.67±0.58 in the normal group, 10.67±1.53 in the PBS-treated group, and 5.33±1.53 in the refined UMSCs-exosomes-treated group (FIG. 7D). The refined UMSCs-exosomes-treated group exhibited a significant 4.50-fold reduction in TUNEL-positive cells compared to the PBS-treated group (p=0.011).

Furthermore, additional investigations were conducted to identify relevant apoptosis markers, including B cell lymphoma-2 (Bcl-2), Bcl-2 associated X (BAX), and cleaved caspase-3 (FIG. 7E). Immunoblot analysis of retinal proteins revealed that the level of the anti-apoptotic protein Bcl-2 increased by 1.77-fold in the refined UMSCs-exosomes-treated group compared to the PBS-treated group (p=0.001). Conversely, the levels of the pro-apoptotic proteins BAX and cleaved caspase-3 in the refined UMSCs-exosomes-treated group decreased by 1.20-fold (p=0.017) and 1.23-fold (p=0.02), respectively, compared to the PBS-treated group (FIG. 7F).

In summary, these findings indicate that treatment with refined UMSCs-exosomes helps maintain RGC density by preventing apoptosis, thereby offering neuroprotective effects in the retina.

### Example 5: Refined UMSCs-Exosomes Treatment Attenuates ONH Edema and Preserves RNFL Thickness

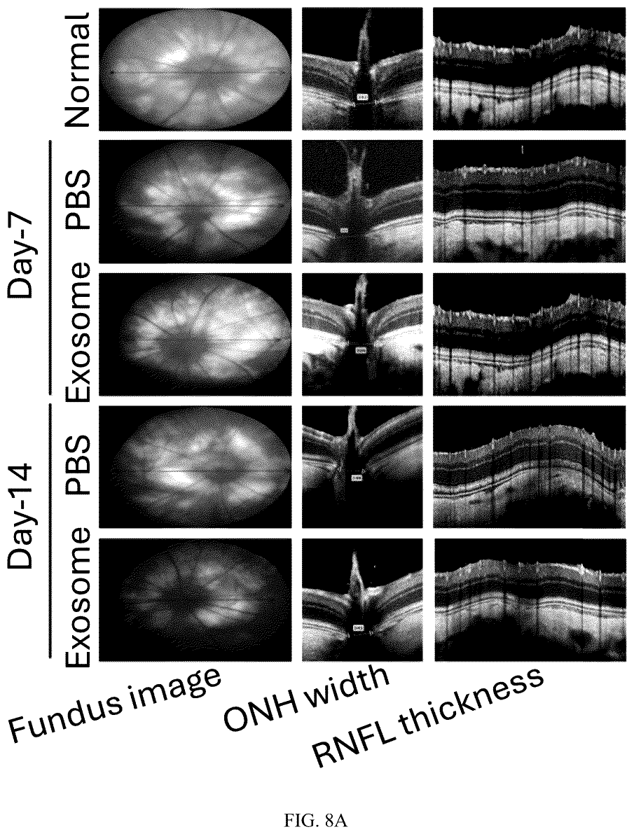

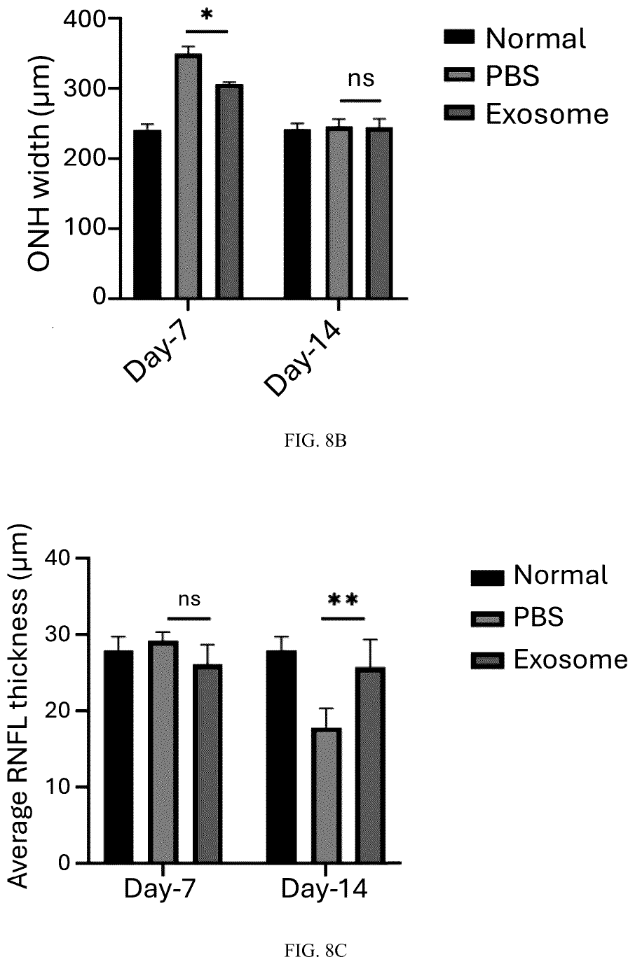

Optic nerve head (ONH) edema and average retinal nerve fiber layer (RNFL) thickness were assessed by optical coherence tomography (OCT) (FIG. 8A). At day 7 post-optic nerve crush (ONC), the ONH width was measured to be 348.5±8.9 μm in the PBS-treated group, showing a 1.45-fold increase compared to the normal group (240.7±8.1 μm). In the refined UMSCs-exosomes-treated group, ONH width was 305.7±2.9 μm, indicating a 1.27-fold decrease compared to the PBS-treated group (p<0.05).

However, the ONH edema completely recovered by day 14 post-ONC (FIG. 8B). On the other hand, at day 7, the average RNFL thickness was 29.13±1.17 μm in the PBS-treated group and 26.07±2.54 μm in the UMSCs-exosomes-treated group, with no significant difference compared to the normal group (27.9±1.8 μm). However, at day 14, RNFL thickness was measured at 17.8±2.48 μm in the PBS-treated group, indicating a 1.22-fold decrease compared to the normal group, and 25.64±3.69 μm in the UMSCs-exosomes-treated group, representing a 1.50-fold increase compared to the PBS-treated group (FIG. 8C). Overall, these data suggest that refined UMSCs-exosomes treatment improves RNFL thickness during disease progression.

### Example 6: Refined UMSCs-Exosomes Treatment Suppresses Macrophage Infiltration in the Optic Nerve

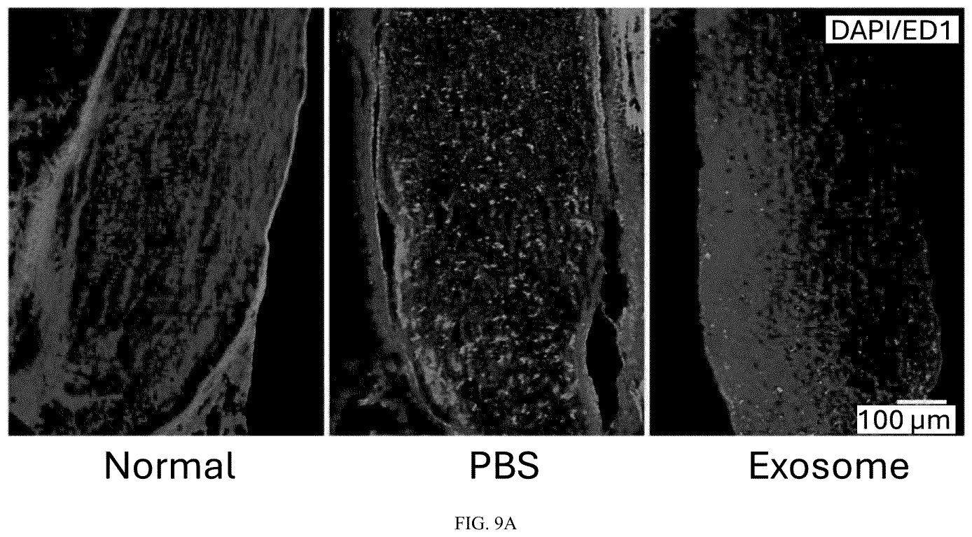

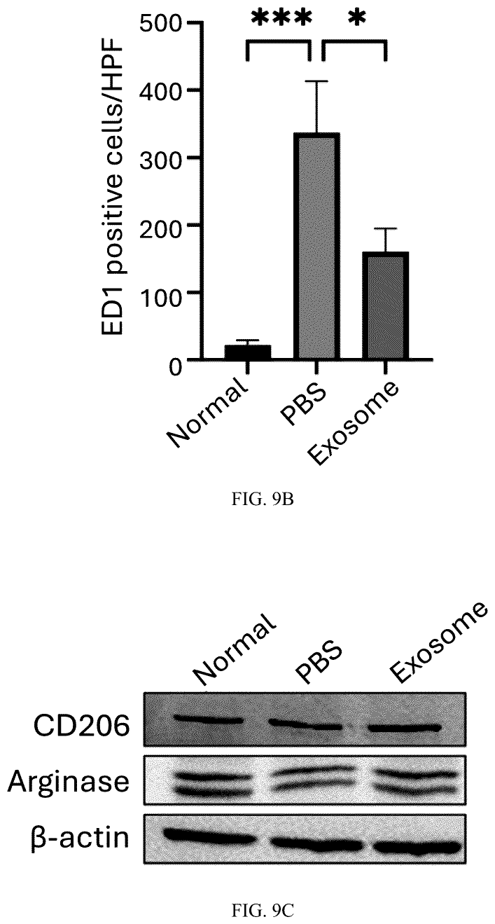

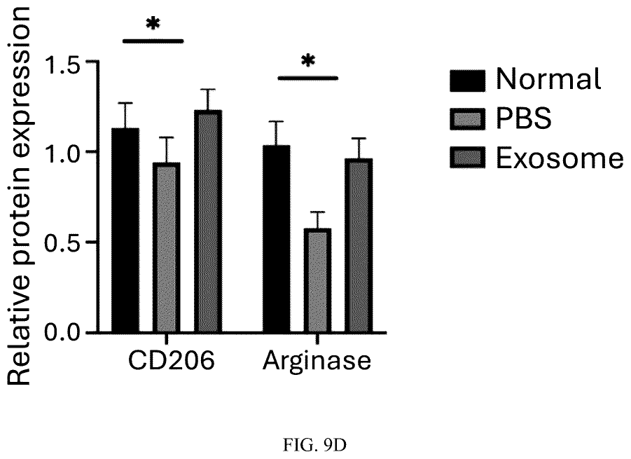

After optic nerve crush (ONC), resident microglia become activated, and blood-borne macrophages are recruited to the injury site. ED1 (CD68) antibody was used to immunostain the activated macrophages, revealing ED1-positive cell counts per HPF of 15.6±2.2, 336.7±76.54, and 106.7±34.27 in the normal, PBS-treated, and refined UMSCs-exosomes-treated groups, respectively (FIGS. 9A and 9B). The refined UMSCs-exosomes-treated group exhibited a 2.1-fold reduction in ED1-positive cells compared to the PBS-treated group (p=0.01).

The expression of M2 inflammatory markers, arginase and the mannose receptor (CD206), was analyzed in the optic nerve at day 14 post-optic nerve crush. Immunoblot analysis revealed a 1.7-fold increase in arginase protein expression (p=0.010) and a 1.3-fold increase in CD206 expression (p=0.004) in the refined UMSCs-exosomes-treated group compared to the PBS-treated group (FIGS. 9C and 9D). These findings suggest that refined UMSCs-exosomes treatment diminished macrophage infiltration into the optic nerve, while elevating the amount of M2 inflammatory markers after optic nerve crush.

### Example 7: Refined UMSCs-Exosomes Treatment Reduces Inflammation in the Optic Nerve

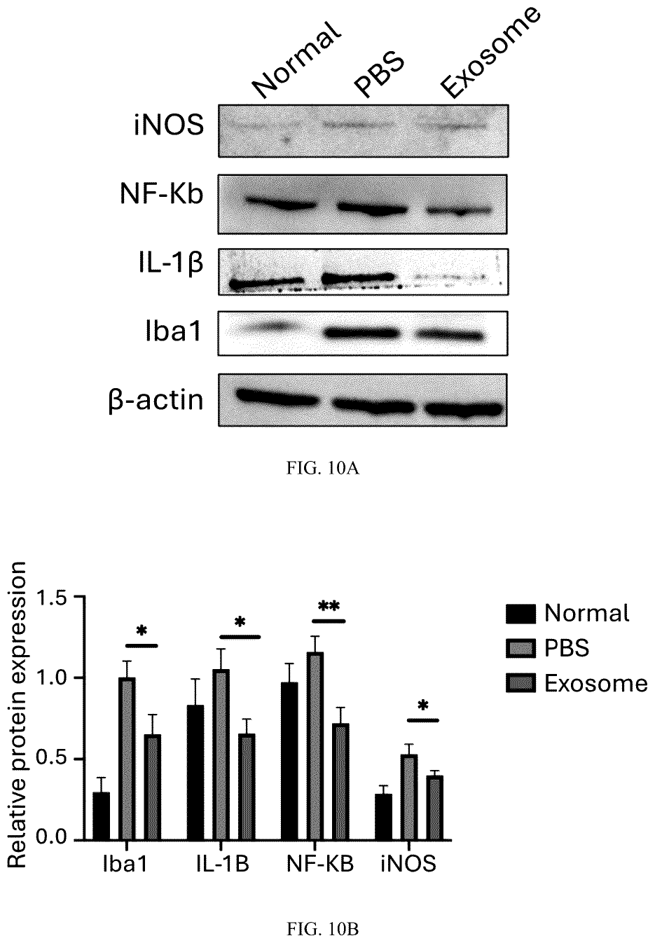

In the context of optic nerve crush (ONC), various inflammatory markers were examined to evaluate the effects of refined UMSCs-exosomes. Treatment with refined UMSCs-exosomes significantly inhibited the levels of the inflammatory cytokines IL-1β and iNOS by 1.60-fold (p<0.05) and 1.61-fold (p=0.04), respectively, compared to the PBS-treated group (FIGS. 10A and 10B). This indicates that refined UMSCs-exosomes treatment effectively decreases the inflammatory response in optic nerve (ON) tissue after ON crush.

Additionally, the protein expressions of Iba1, a marker for activated microglia and macrophages, and NF-kB, a key regulator of immune response and inflammation, were evaluated. Results showed that Iba1 and NF-kB levels were reduced by 1.53-fold (p<0.05) and 1.32-fold (p=0.03), respectively, in the refined UMSCs-exosomes-treated group compared to the PBS-treated group (FIGS. 10A and 10B). These findings suggest that refined UMSCs-exosomes treatment significantly reduces inflammation in ON tissue following ON crush.

### Example 8: Refined UMSCs-Exosomes Treatment Increases Antioxidant Activity in the Retina

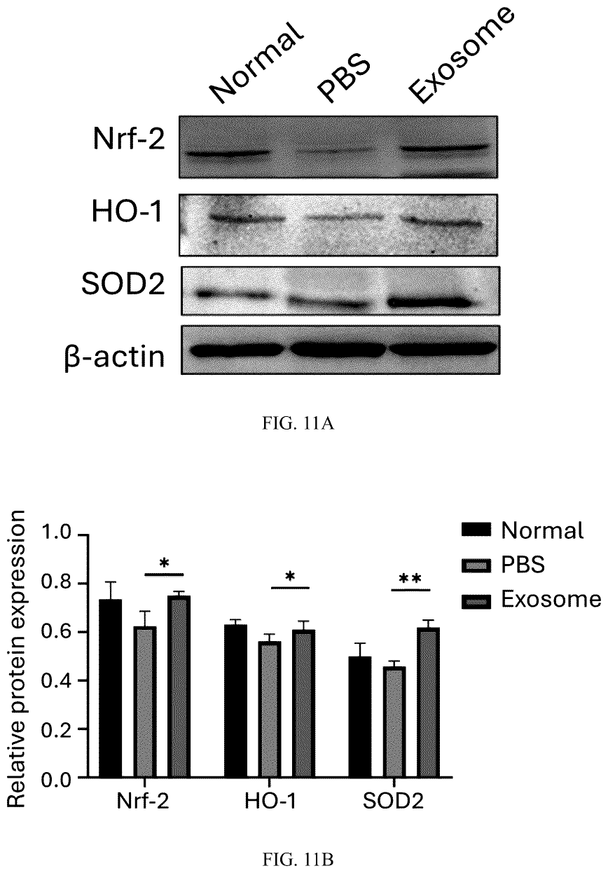

Oxidative stress is a key factor in the development of traumatic optic neuropathy (TON) as it can initiate cell death. To evaluate the impact of refined UMSCs-exosomes on antioxidative stress, protein expression levels of Nrf2, HO-1, and SOD2 were analyzed using western blotting. The treatment with refined UMSCs-exosomes resulted in significant increases in the levels of Nrf2 by 1.2-fold (p=0.02), HO-1 by 1.09-fold (p=0.03), and SOD2 by 1.35-fold (p=0.006) compared to the PBS-treated group (FIGS. 11A and 11B). These findings suggest that refined UMSCs-exosomes enhance the antioxidative stress response in retinal tissues following optic nerve crush.

### Example 9: Refined UMSCs-Exosomes Promote Antioxidative Activity in the Retina Through the Activation of the pAkt/ERK/Nrf2 Pathway

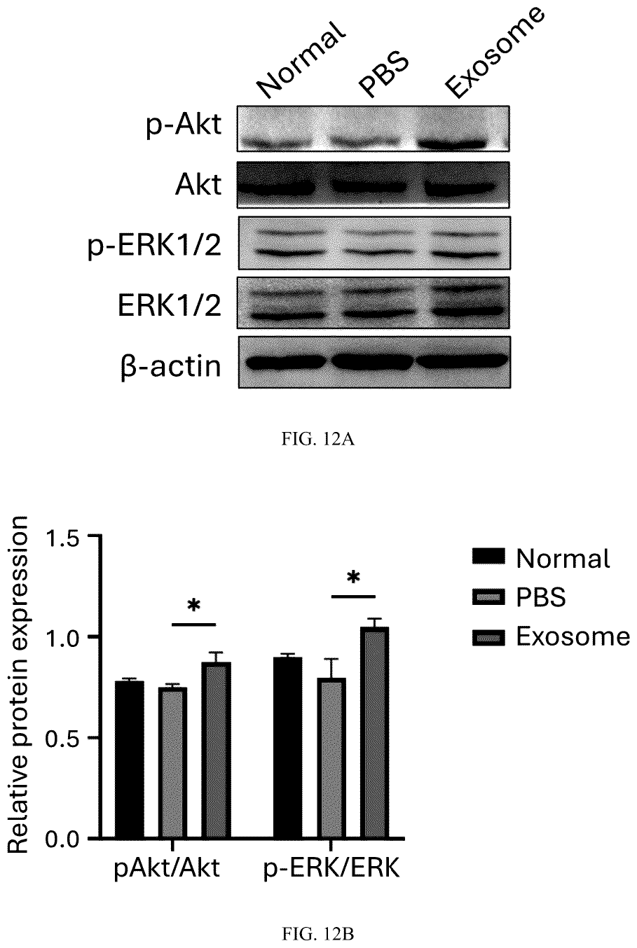

In this example, it was investigated to find out whether refined UMSCs-exosomes enhance antioxidative activity in the retina through the pAkt/ERK/Nrf2 pathway. Western blot analysis revealed a significant increase in phosphorylated levels of Akt by 1.17-fold (p=0.02) and ERK by 1.3-fold (p=0.04) following the treatment of refined UMSCs-exosomes compared to the PBS-treated group (FIGS. 12A and 12B). These findings suggest that refined UMSCs-exosomes activate the pAkt/Akt and ERK pathways, thereby triggering Nrf2 activation and promoting antioxidative responses in retinal cells.

While some of the embodiments of the present disclosure have been described in detail in the above, it is, however, possible for those of ordinary skill in the art to make various modifications and changes to the embodiments shown without substantially departing from the teaching of the present disclosure. Such modifications and changes are encompassed in the scope of the present disclosure as set forth in the appended claims.

What is claimed is:

1. A method for preventing or treating optic neuropathy in a subject in need thereof, comprising administering to said subject a therapeutically effective amount of a composition comprising an exosome isolated from a mesenchymal stem cell and an excipient thereof, wherein the isolated exosome undergoes a refinement procedure.

2. The method of claim 1, wherein the refinement procedure comprises an ultrafiltration.

3. The method of claim 2, wherein the ultrafiltration is carried out with a centrifugal filter device with a 100,000 molecular weight cutoff membrane.

4. The method of claim 1, wherein the refinement procedure comprises at least one cycle of concentration and centrifugation.

5. The method of claim 4, wherein the centrifugation is carried out at from 1,000×g to 20,000×g.

6. The method of claim 4, wherein the concentration is at least 5-times concentration.

7. The method of claim 4, wherein the refinement procedure comprises reconstitution with Hank's balanced salt solution after concentration.

8. The method of claim 1, wherein the refined exosome reduces toxicity associated with administration of an exosome to an eye.

9. The method of claim 8, wherein the administration is intravitreal injection.

10. The method of claim 1, wherein the refined exosome preserves retinal function in an eye.

11. The method of claim 1, wherein the refined exosome preserves visual function in an eye.

12. The method of claim 1, wherein the refined exosome provides a neuroprotective effect to a retina in an eye.

13. The method of claim 1, wherein the refined exosome suppresses macrophage infiltration in an optic nerve in an eye.

14. The method of claim 1, wherein the refined exosome reduces inflammation in an optic nerve in an eye.

15. The method of claim 1, wherein the refined exosome increases antioxidant activity in a retina in an eye.

16. The method of claim 1, wherein the optic neuropathy is ischemic optic neuropathy, inflammatory optic neuropathy, compressive optic neuropathy, infiltrative optic neuropathy, traumatic optic neuropathy, mitochondrial optic neuropathy, nutritional optic neuropathy, toxic optic neuropathy, hereditary optic neuropathy, radiation-induced optic neuropathy, optic nerve atrophy, glaucoma, or optic neuritis.

17. A method for preparing the composition of claim 1, comprising:
obtaining an exosome isolated from a mesenchymal stem cell; and
refining the isolated exosome,
wherein the refining comprises at least one of ultrafiltration, reconstitution, centrifugation, and concentration.

18. A composition comprising the exosome prepared by the method of claim 17 and an excipient thereof.
